(19) United States
(12) Patent Application Publication
(10) Pub. No.: US 2024/0118351 A1
(43) Pub. Date: Apr. 11, 2024

(54) SCANNER AND IMAGE PROCESSING SYSTEM THAT RECOGNIZE GENUINE CABLE

(71) Applicant: MEDIT CORP., Seoul (KR)

(72) Inventors: Hyeong Seok KIM, Seoul (KR); Dong Hoon LEE, Seoul (KR)

(73) Assignee: MEDIT CORP., Seoul (KR)

(21) Appl. No.: 18/275,316

(22) PCT Filed: Jan. 28, 2022
(86) PCT No.: PCT/KR2022/001568

§ 371 Date: Aug. 1, 2023

(30) Foreign Application Priority Data
Feb. 2, 2021 (KR) 10-2021-0014728
Dec. 3, 2021 (KR) 10-2021-0172260

(51) Int. Cl. G01R 31/55; G01R 31/58

(52) U.S. Cl. CPC G01R 31/55; G01R 31/58

(57) ABSTRACT

Provided is a scanner including a camera for obtaining image data by scanning an object, a communication interface including a port to which a cable for performing communication with an external device is connected, a processor, and a memory storing instructions executable by the processor, wherein the processor is configured to, by executing the instructions, transmit a signal to the cable connected to an image processing device receiving the image data, to obtain recognition information indicating whether the cable is genuine, based on a feedback signal from the cable in response to the signal, and to transmit the recognition information to the image processing device via the cable.

### TECHNICAL FIELD

The disclosure relates to a scanner and an image processing system. More particularly, the disclosure relates to recognizing whether a cable connected to a scanner is genuine and providing a recognition result to an image processing system.

### BACKGROUND ART

A scanner may be inserted into an oral cavity of a patient and obtain image data within the oral cavity. The scanner may transmit the image data to an image processing device. In this case, the scanner may transmit the image data to the image processing device through a cable connecting the scanner to the image processing device. When the image data is transmitted in a state in which a genuine cable is not connected to the scanner, partial data of the image data may be missing or the cable may be unstable, and thus, transmission of the image data may not be carried out normally.

Accordingly, there is a need for a technique of recognizing whether a cable connected to a scanner is a genuine cable, and inducing the use of a genuine cable when a non-genuine cable is used.

### DISCLOSURE

### Technical Problem

Disclosed embodiments are to recognize whether a cable connected to a scanner in the scanner is genuine, and provide a recognition result to an image processing device.

Disclosed embodiments are to, when a cable is not genuine, record log information about the cable in a scanner or limit an operation of the scanner.

Disclosed embodiments are to display, on an image processing device, notification information notifying use of a cable identified as being non-genuine.

### Technical Solution

According to an aspect, provided is a scanner including a camera for obtaining image data by scanning an object, a communication interface including a port to which a cable for performing communication with an external device is connected, a processor, and a memory storing instructions executable by the processor, wherein the processor is configured, by executing the instructions, to transmit a signal to the cable connected to an image processing device which receives the image data, and to obtain recognition information indicating whether the cable is genuine, based on a feedback signal from the cable in response to the signal.

The processor may be further configured, by executing the instructions, based on the feedback signal being received from the cable, to obtain verification information indicating that the cable is identified to be genuine, and based on the feedback signal not being received from the cable, to obtain error information indicating that the cable is identified to be non-genuine.

The processor may be further configured to, by executing the instructions, based on the cable being identified to be non-genuine, record log information about the cable in the memory.

The log information may include at least one from among information on a time during which the cable is connected, and information on a time during which connection to the cable is released.

The processor may be further configured to, by executing the instructions, based on the cable being identified to be non-genuine, at least one operation from among an operation of controlling a preset function of the scanner, an operation of controlling a voltage booster circuit for boosting a voltage of the scanner, and an operation of controlling at least one element within the scanner.

The scanner may further include a genuine cable connectable to the port, wherein the genuine cable may include an identifier in response to a signal transmitted via the communication interface.

The processor is further configured, by executing the instructions, to obtain, from a power supply device for supplying power to the scanner, the recognition information indicating whether a first cable which connects the port to a first port of the power supply device, is genuine, and to transmit the recognition information to the image processing device via a second cable connecting a second port of the power supply device to the image processing device.

The processor may be further configured to, by executing the instructions, perform at least one operation from among an operation of transmitting the recognition information to the image processing device via the cable and an operation of outputting the recognition information.

According to another aspect, an operating method of a scanner includes transmitting a signal to a cable connected to an image processing device which receives image data obtained from the scanner, and obtaining recognition information indicating whether the cable is genuine, based on a feedback signal from the cable in response to the signal.

The obtaining of the recognition information based on the feedback signal may include, based on the feedback signal being received, obtaining verification information indicating that the cable is identified to be genuine, and based on the feedback signal not being received, obtaining error information indicating the cable is identified to be non-genuine.

The method of operating the scanner may further include, based on the cable being identified to be non-genuine, recording log information about the cable, wherein the log information may include at least one from among information on a time during which the cable is connected and information on a time during which connection of the cable is released.

The method of operating the scanner may further include, based on the cable being identified to be non-genuine, performing at least one operation from among an operation of controlling a preset function of the scanner, an operation of controlling a voltage booster for boosting a voltage of the scanner, and an operation of controlling at least one element within the scanner.

According to another aspect, A computer program stored in a computer-readable medium so as to be coupled to an image processing device and executed includes an instruction to identify connection of a cable used to perform communication with a scanner, an instruction to receive, from the scanner, recognition information indicating whether the cable is genuine, and an instruction to display, when the recognition information indicates that the cable is not genuine, notification information indicating that the cable is not genuine.

The computer program may further include an instruction to receive image data of an object obtained from the scanner, and an instruction to display the notification information on an image corresponding to the image data.

The instruction to display the notification information may include at least one instruction from among an instruction to display the notification information in a pop-up window, an instruction to display the notification information in an area other than an area of interest included in an image corresponding to image data of an object, and an instruction to display a watermark indicating the notification information.

The notification information may include at least one from among information indicating that the cable is non-genuine and information indicating that replacement of the cable is requested with a genuine cable.

### Advantageous Effects

According to s embodiments, a scanner may recognize whether a cable connected to a scanner is genuine, and provide a result of the recognition to an image processing device.

According to disclosed embodiments, when a cable is not genuine, log information about a cable may be recorded by the scanner, or an operation of the scanner may be limited.

According to disclosed embodiments, the image processing device may display notification information notifying use of a cable identified as non-genuine.

According to disclosed embodiments, a user may be notified as to whether the cable being used is genuine, and when the cable being used is not genuine, the user may induce a genuine cable to be used.

According to disclosed embodiments, an environment in which the scanner and the image processing device are stably used may be provided to the user.

### DESCRIPTION OF DRAWINGS

The present disclosure may be easily understood from the following detailed description and a combination of the accompanying drawings, and reference numerals denote structural elements.

FIG. 1 is a conceptual diagram for describing a process of transmitting, from a scanner to an image processing device, recognition information indicating whether a cable is genuine, and displaying notification information according to the recognition information by the image processing device, according to an embodiment.

FIG. 2 is a block diagram illustrating a configuration of a scanner according to an embodiment.

FIG. 3 is a flowchart of an operation method of a scanner according to an embodiment.

FIG. 4A is a diagram for describing a process of identifying a structure of a cable and genuineness of the cable, according to an embodiment.

FIG. 4B is a diagram for describing a process of identifying a structure of a cable and genuineness of the cable, according to another embodiment.

FIG. 5 is a diagram for describing a process of identifying genuineness of a cable when a power supply device is connected to a scanner, according to an embodiment.

FIG. 6 is a block diagram of a configuration of an image processing device according to an embodiment.

FIG. 7 is a flowchart of an operation method of an image processing device according to an embodiment.

FIGS. 8A to 8C illustrate a screen displaying notification information indicating that a cable is not genuine, when it is identified that the cable is not genuine, according to an embodiment.

FIG. 9 is a flowchart of an operation method of an image processing system according to an embodiment.

FIG. 10 is a diagram for describing instructions stored in a computer system stored in a computer-readable medium, according to an embodiment.

### BEST MODE

Provided is a scanner including a camera for scanning an object and obtaining image data; a communication interface including a port to which a cable for performing communication with an external device is connected; a processor; and a memory for storing instructions executable by the processor, wherein the processor, by executing the instructions, transmits a signal to a cable connected to an image processing device receiving the image data, obtains recognition information indicating whether the cable is genuine, on the basis of a feedback signal from the cable in response to the signal, and transmits the recognition information to the image processing device through the cable.

### MODE FOR INVENTION

Various embodiments are described below in detail with reference to the drawings. Embodiments described below may be modified in various different forms and worked. To more clearly describe features of embodiments, detailed descriptions of matters well known in a person having ordinary skill in the art to which the following embodiments pertain are omitted.

Meanwhile, in the present specification, when an element is “connected” to another element, it includes not only a case of being directly connected, but also a case of being connected with another element therebetween. In addition, when an element “includes” another element, it means that other elements may be further included rather than excluding the other elements, unless otherwise specified.

In addition, a term including an ordinal number, such as “first” or “second”, used in the present specification may be used to describe various elements, but the elements are not to be limited by the terms described above. The terms are used only to distinguish one element from another element.

In the specification as a whole, a “scanner” may denote a device for obtaining image data related to an object. The scanner may denote a scanner for obtaining image data related to an oral cavity for use in intraoral treatment. For example, the intraoral scanner may be an intraoral scanner having a shape that may be inserted into an oral cavity. Here, it is general that the intraoral scanner has a portable shape that may be held by one hand, and the intraoral scanner may be referred to as a hand-held type scanner. Alternatively, the scanner may be a table-type scanner that may be used for dental treatment.

In addition, the “scanner” may use a cable to perform communication with an external device. The scanner may include at least one port to connect to the cable. The scanner and the external device may be directly or indirectly connected by the cable. For example, when the scanner and the external device are directly connected by the cable, it may mean that a first terminal of the cable is connected to the scanner and a second terminal of the cable is connected to the external device. For example, when the scanner and the external device are indirectly connected by the cable, it may mean that the scanner and the external device are connected to the cable via an intermediate medium. For example, the intermediate medium may be a power supply device.

A “cable” may refer to one that is composed of a wire or optical fiber inside a sheath. The wire or the optical fiber in the cable may be insulated. For example, a component of the wire may be copper, aluminum, or the like. The cable may be used to exchange data between the scanner and the external device. In addition, the cable may be used to receive power from the outside.

An “object” is one that is subject of photographing, and may include a person, an animal, or a portion thereof. For example, the object may include a body part (an organ or the like), an artificial structure attachable or insertable onto/into the object, a phantom, or the like. For example, the object may include at least a partial area of a tooth, a gingiva, and an oral cavity, and/or an artificial structure insertable into the oral cavity (e.g., an orthodontic device including a bracket and a wire, a dental restoration including an implant, an artificial tooth, an inlay, and an onlay, orthodontic aids inserted into the oral cavity, a tooth or a gingiva to which artificial structure s re attached, and the like.

The “image processing device” may denote a device for receiving image data from a scanner and performing at least one operation from among generating, processing, displaying, and transmitting an image corresponding to the image data For example, the image processing device may be a computing device, such as a laptop computer, a desktop computer, a personal digital assistant (PDA), a tablet personal computer (PC), a smartphone, or the like, and is not limited to the examples described above.

For example, the image processing device may generate at least one of information necessary for intraoral diagnosis, an image representing an oral cavity, and a model used in intraoral treatment based on image data received from a scanner, and display the generated information and image.

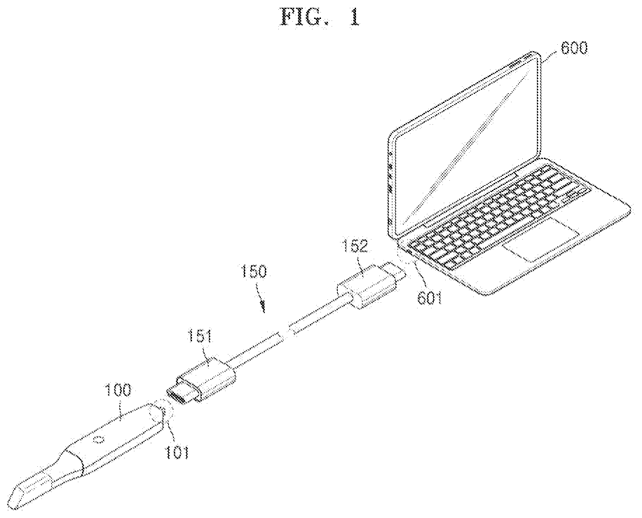

FIG. 1 is a conceptual diagram for describing a process of transmitting, from a scanner to an image processing device, recognition information indicating whether a cable is genuine, and displaying notification information on the image processing device according to the recognition information.

Referring to FIG. 1, an image processing system may include a scanner 100 for obtaining image data by scanning an object, and transmitting the image data to an image processing device 600, and the image processing device 600 for receiving the image data from the scanner 100 and displaying an image corresponding to the image data. A cable 150 may be used to transmit the image data from the scanner 100 to the image processing device 600.

Because the scanner 100 transfers thousands of or tens of thousands of pieces of image data to the image processing device 600 in a short time, when a non-genuine cable is used instead of a genuine cable, part of image data may be missing. In addition, a state of the cable may become unstable so that power may be turned off. In addition, a speed at which the image data is processed by the image processing device 600 may also be slowed down. Accordingly, it is necessary to use a genuine cable for the cable 150 used to transmit the image data from the scanner 10 to the image processing device 600. The scanner 100 may identify whether the cable 150 connected to the scanner 100 and the image processing device 600 is genuine, and transfer a result of the identification to the image processing device 600. The image processing device 600 may display the identification result.

Referring to FIG. 1, a first terminal 151 of the cable 150 may be connected to a port 101 of the scanner 100, and a second terminal 152 may be connected to a port 601 of the image processing device 600. The scanner 100 may transmit a signal to the cable 150 and identify whether a feedback signal in response to the signal is received from the cable 150. The scanner 100 may identify whether the cable 150 is genuine based on the feedback signal, and obtain recognition information indicating whether the cable 150 is genuine. The scanner 100 may transmit the recognition information to the image processing device 600 through the cable 150. For example, when the recognition information indicates that the cable 150 is not genuine, the image processing device 600 may display notification information indicating that the cable 150 is not genuine.

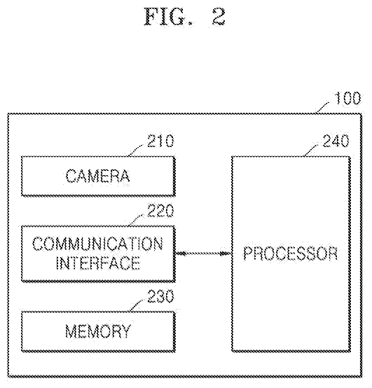

FIG. 2 is a block diagram of a configuration of a scanner according to an embodiment.

Referring to FIG. 2, the scanner 100 may include a camera 210, a communication interface 220, a memory 230, and a processor 240. However, not all of the elements shown are essential elements. The scanner 100 may be implemented by more elements than shown, and the scanner 100 may also be implemented by less elements. Hereinafter, the elements described above are discussed.

The camera 210 may obtain image data of an object by photographing the object. For example, the camera 210 may obtain at least one piece of two-dimensional image data, and generate three-dimensional image data based on the at least one obtained piece of two-dimensional image data. For example, the camera 210 may obtain at least one piece of two-dimensional image data of an oral cavity by photographing the oral cavity. For example, when the oral cavity is photographed, it may mean photographing not only the oral cavity itself, but also artificial objects and/or other objects representing the oral cavity or related to the oral cavity.

The communication interface 220 may perform communication with an external device. For example, the communication interface 220 may be connected by wire to perform communication with the external device. The communication interface 220 may have a port to which a cable for performing communication with the external device is connected. For example, the port may be designed in a structure to which a terminal of the cable may be coupled.

In addition, the communication interface 220 may perform communication with the external device wirelessly. The communication interface 220 may include a communication module used for wireless communication. For example, the communication module may be in the form of a chipset, and may be a sticker/barcode (e.g., a sticker including an NFC tag) or the like including information necessary for communication.

In addition, the communication interface 220 may support at least one of Wireless LAN, Wireless Fidelity (Wi-Fi), Wi-Fi Direct (WFD), Bluetooth, Bluetooth Low Energy (BLE), Wired Lan, Near Field Communication (NFC), Zigbee, infrared Data Association (IrDA), 3G, 4G, and 5G.

The communication interface 220 may transmit, via a cable, at least one piece of two-dimensional image data or three-dimensional image data to the image processing device 600.

The memory 230 may store software or programs. For example, the memory 230 may store a program for controlling an operation of the scanner 100. In addition, the memory 230 may store image data obtained from the camera 210. In addition, the memory 230 may store recognition information indicating whether a cable is genuine. When the cable is identified to be non-genuine, the memory 230 may store log information about the cable. For example, the log information may include history information about performing scanning by using the non-genuine cable. In another example, the log information may include information about a use time of the cable. Specifically, the use time of the cable may include at least one from among information on a time during the cable is connected to the scanner 100 and information on a time at which the cable releases connection with the scanner 100. In addition, the memory 230 may store at least one instruction for executing an operation method of the scanner 100 for obtaining recognition information whether a cable connected to the scanner 100 is genuine, and transmitting the recognition information to the image processing device 600.

The processor 240 may control overall operations of the scanner 100 and may include at least one processor, such as a CPU or the like. The processor 240 may include at least one processor specialized for each function, or may be a processor in one integrated form.

The processor 240 may execute a program stored in the memory 230, read data or a file stored in the memory 230, or s tore a new file in the memory 230. The processor 240 may execute instructions stored in the memory 230.

The processor 240 may transmit a signal to a cable connected to the scanner 100 via the communication interface 220. In this case, the cable may connect the scanner 100 to the image processing device 600. Specifically, a first terminal of the cable may be connected to a port of the scanner 100, and a second terminal of the cable may be connected to a port of the image processing device 600. The cable may be operated to exchange data between the scanner 100 and the image processing device 600.

The processor 240 may obtain recognition information indicating whether the cable is genuine, based on a feedback signal from the cable in response to a signal. For example, the processor 240 may identify, after transmitting a signal to the cable, whether the feedback signal is received from the cable within a preset time. For example, the feedback signal may be a signal indicating that the cable is genuine.

For example, when the feedback signal is received, the processor 240 may identify that the cable is genuine, and obtain verification information indicating that the cable is identified to be genuine. On the other hand, when the feedback signal is not received, the processor 240 may identify that the cable is non-genuine, and obtain error information indicating that the cable is identified to be non-genuine. The processor 240 may transmit recognition information to the image processing device 600 through the communication interface 220. For example, when it is identified that the cable is non-genuine, the processor 240 may transmit an error code indicating that the cable is non-genuine, to the image processing device 600 via the communication interface 220.

Meanwhile, when the cable is identified to be non-genuine, the processor 240 may record log information about the cable in the memory 230. For example, the log information may include at least one from among information on a time during which the cable is connected and information on a time during which the connection of the cable is released. By recording the log information in the scanner 100, it may be identified that the user has used a non-genuine cable.

When the cable is identified to be non-genuine, the processor 240 may perform an operation of controlling all functions or a preset function of the scanner 100. For example, the processor 240 may change at least one of an operating condition and an operation range for all functions or a preset function. For example, when the cable is non-genuine, image data may be missing in a transmission process for the image data, and thus, the processor 240 may control a shooting mode to be deactivated, the shooting mode corresponding to high quality or high performance from among shooting modes provided by the scanner 100. In addition, the processor 240 may control the scanner 100 is activated only during a preset time.

When the cable is identified to be non-genuine, the processor 240 may perform an operation of controlling a booster circuit that boosts up a voltage of the scanner 100. The booster circuit may increase a low voltage within a preset range to a voltage within a spec range of the scanner 100. For example, when the cable is identified as non-genuine, the processor 240 may perform a control operation for deactivating the booster circuit or limiting a voltage increase. When the voltage booster circuit is deactivated or the voltage increase is limited, the scanner 100 may not operate normally.

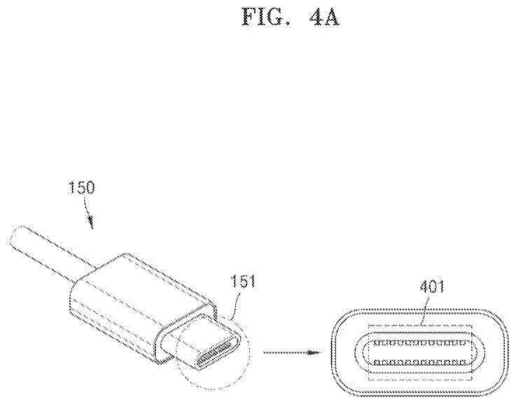

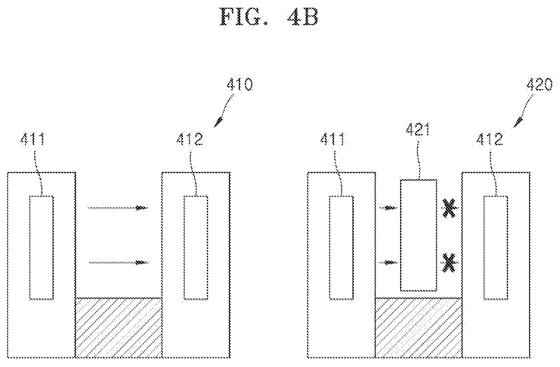

Meanwhile, when the cable is genuine, the genuine cable may include an identifier in response to a signal transmitted via the communication interface 220. A structure of a genuine cable and a process of identifying genuineness are described with reference to FIGS. 4A and 4B.

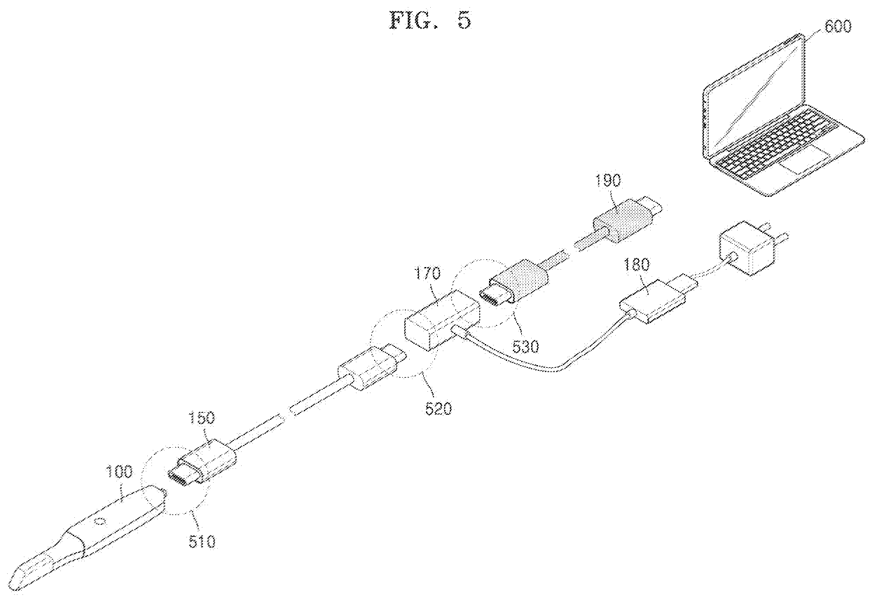

For example, when the image processing device 600 connected to the scanner 100 via a cable is not capable of sufficiently supplying power to the scanner 100, a power supply device for a power supply may be connected to the scanner 100. For example, the power supply device may receive power from the outside and supply power to the scanner 100. For example, the power supply device may include a hub and an adapter. For example, the hub may include a first port connected to a first cable connected to a port of the scanner 100, a second port connected to a second cable connected to the image processing device 600, and a third port connected to the adapter. For example, the processor 240 may obtain, from the power supply device, recognition information indicating whether the first cable is genuine, and transmit the recognition information to the image processing device 600 via a second cable. An operation of a case in which the power supply device is connected to the scanner 100 is described with reference to FIG. 5.

For example, the scanner 100 may output the recognition information. For example, when an LED is present in the scanner 100, the processor 240 may output the recognition information through the LED. For example, the processor 240 may output the recognition information based on at least one of an on/off state of the LED and a color of light emitted from the LED.

For example, when the recognition information indicates verification information indicating that the cable is identified to be genuine, the processor 240 may control the LED to turn off the LED or to output light of a first color. For example, when the recognition information indicates error information indicating that the cable is identified to be non-genuine, the processor 240 may control the LED to turn on the LED or to output light of a second color.

For example, when a display is present in the scanner 100, the processor 240 may output the recognition information through the display.

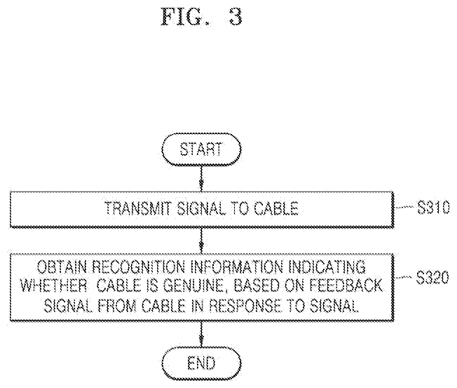

FIG. 3 is a flowchart of an operation method of a scanner according to an embodiment.

Referring to FIG. 3, in operation S310, when a cable is connected to the scanner 100, the scanner 100 may transmit, to the cable, a signal for identifying whether the cable is genuine. For example, when the cable is connected to the scanner 100, a power of the cable may be switched from an off state to an on state. In addition, the cable is connected to the scanner 100, and according to a manipulation input of a user, the power of the cable may be switched from the off state to the on state. In addition, the cable is connected to the scanner 100, and after log information is recorded in the scanner 100, the power of the cable may be in the on state.

In another example, the scanner 100 may identify that the cable is connected to the scanner 100 and the image processing device 600, and transmit, to the cable, a signal for identifying whether the cable is genuine.

In operation S320, the scanner 100 may recognition information indicating whether the cable is genuine, based on a feedback signal from the cable in response to a signal.

For example, the scanner 100 may identify, after transmitting the signal to the cable, whether a feedback signal is received from the cable within a preset time.

For example, when the feedback signal is received from the cable, the scanner 100 may obtain verification information indicating that the cable is identified to be genuine. On the other hand, when the feedback signal is not received from the cable, error information indicating that the cable is identified to be non-genuine may be obtained.

For example, the scanner 100 may transmit the recognition information to the image processing device 600 via the cable.

For example, the scanner 100 may output the recognition information. For example, when an LED is present in the scanner 100, the scanner 100 may output the recognition information through the LED. For example, the scanner 100 may output the recognition information based on at least one of an on/off state of the LED or a color of light emitted from the LED.

For example, when the recognition information indicates verification information indicating that the cable is identified to be genuine, the scanner 100 may control the LED to turn off the LED or to output light of a first color. For example, when the recognition information indicates error information indicating that the cable is identified to be non-genuine, the scanner 100 may control the LED to turn on the LED or to output light of a second color.

For example, when a display is present in the scanner 100, the scanner 100 may output the recognition information through the display.

Meanwhile, when the cable is identified to be non-genuine, the scanner 100 may record log information about the cable in a memory. For example, the log information may include at least one from among information on a time during which the cable is connected to the scanner 100 and information on a time during which connection between the cable and the scanner 100 is released.

For example, when the cable is identified to be non-genuine, the scanner 100 may perform at least one operation from among an operation of controlling a preset function of the scanner 100 and an operation of controlling a voltage booster circuit that increases a voltage of the scanner 100.

For example, when the cable is a genuine cable, the genuine cable may include an identifier for responding to a signal transmitted through a communication interface of the scanner 100.

In addition, when the image processing device 600 does not support a power supply to the scanner 100, the scanner 100 may be connected to a power supply device. The power supply device may include a first port connected to a first cable connected to the scanner 100, and a second port connected to a second cable connected to the image processing device 600. The power supply device may obtain recognition information indicating whether the first cable connected to the scanner 100 is genuine, and transmit the recognition information to the image processing device 600 via the second cable. A process of identifying genuineness of a cable by a power supply device, when the power supply device is connected to the scanner 100, is described with reference to FIG. 5.

FIG. 4A is a diagram for describing a process of identifying a structure of a cable and a process of identifying genuineness of the cable, according to an embodiment.

FIG. 4A is a diagram for describing a terminal of the cable. As shown in FIG. 4A, in a terminal 151 of the cable, a structure that may be connected to a port of the scanner 100 may be provided. An identifier 401 for identifying whether the cable is genuine may be included inside the terminal 151 of the cable. For example, the identifier 401 may be a pin or a circuit.

For example, when the scanner transmits a signal to the cable, the identifier 401 may transmit a feedback signal to the scanner 100 in response to the signal. Because the identifier 401 is not provided when the cable is non-genuine, the scanner 100 may not receive a feedback signal in response to the signal. Accordingly, the scanner 100 may identify whether the cable is genuine according to the feedback signal. The scanner 100 may transmit, to the image processing device 600, recognition information indicating whether the cable is genuine.

FIG. 4B is a diagram for describing a structure of a cable and a process of identifying genuineness of the cable, according to another embodiment.

An image 410 of the FIG. 4B is a view illustrating a terminal of the cable before the cable is connected to a port of the scanner 100. The terminal of the cable may have a structure that may be coupled to the port of the scanner 100. In addition, identifiers 411 and 412 for identifying that the cable is genuine may be included on surfaces of the terminal of the cable. For example, the identifier 411 may be included on a first surface of the terminal of the cable, and the identifier 412 may be included on a second surface of the terminal of the cable. For example, the first surface and the second surface may be surfaces facing each other. For example, the identifiers 411 and 412 may be sensors for detecting the port of the scanner when the terminal of the cable and the port of the scanner 100 are coupled to each other. For example, the identifiers 411 and 412 may be at least one of proximity sensor, a photosensor, a pressure sensor, and a magnetic sensor.

An image 420 of FIG. 4 is a view illustrating the terminal of the cable after the cable is connected to a port 421 of the scanner 100. For example, when the identifiers 411 and 412 are photosensors and before the cable is connected to the port of the scanner 100, referring to the image 410 of FIG. 4B, when the identifier 411 emits infrared light, the identifier 412 may receive the infrared light. On the other hand, after the cable is connected to the port of the scanner 100, referring to the image 420 of FIG. 4B, when the identifier 411 emits infrared light, the infrared light is not able to pass therethrough due to the port 421 of the scanner 100, and thus, the infrared light may not reach the identifier 412. For example, operations of the identifiers 411 and 412 may be initiated after the cable is connected to the port of the scanner 100 and a signal is received from the scanner 100. The cable may obtain values detected by the identifiers 411 and 412 as a feedback signal, and transmit the feedback signal to the scanner 100.

When the cable is non-genuine, the identifiers 411 and 412 are not provided, and thus, the scanner 100 may not receive a feedback signal in response to the signal. Accordingly, the scanner 100 may identify whether the cable is genuine based on the feedback signal. The scanner 100 may transmit, to the image processing device 600, recognition information indicating whether the cable is genuine.

For example, the cable may obtain verification information indicating that the cable is genuine based on the values detected by the identifiers 411 and 412. The cable may transmit the verification information to the scanner 100 or the image processing device 600.

For example, although it is described that the identifiers 411 and 412 are included in the cable, reversely, the identifiers 411 and 412 may be included in the scanner 100, and the port 421 may be included in the cable.

FIG. 5 is a diagram for describing a process of identifying genuineness of a cable, when a power supply device is connected to the scanner 100, according to an embodiment.

Referring to FIG. 5, an image processing system may include the scanner 100 for obtaining image data by scanning an object, and transmitting the image data to the image processing device 600, and the image processing device 600 for receiving the image data from the scanner 100 and displaying an image corresponding to the image data. For example, when the image processing device 600 is not capable of sufficiently supplying power to the scanner 100, a power supply device for a power supply may be connected to the scanner 100. The power supply device may include a hub 170 and an adapter 180. The first cable 150 for connecting the scanner 100 to the hub 170 may be used, and a second cable 190 for connecting the hub 170 to the image processing device 600 may be used.

For example, the hub 170 may include a first port connected to the first cable connected to a port of the scanner 100, a second port connected to the second cable connected to the image processing device 600, and a third port connected to the adapter 180.

The scanner 100 or the hub 170 may perform an operation of identifying whether the first cable 150 is genuine. For example, at a first location 510, the scanner 100 may perform the operation of identifying whether the first cable 150 is genuine. For example, the scanner 100 may transmit a signal to the first cable 150. The scanner 100 may obtain recognition information whether the first cable 150 is genuine, based on a feedback signal from the first cable 150 in response to the signal. The scanner 100 may transmit the recognition information to the image processing device 600. When the recognition information indicates that the first cable 150 is not genuine, the image processing device 600 may display notification information indicating that the first cable 150 is not genuine.

For example, at a second location 520, the hub 170 may perform the operation of identifying whether the first cable 150 is genuine. Specifically, the hub 170 may transmit a signal to the first cable 150. The hub 170 may obtain recognition information indicating whether the first cable 150 is genuine, based on a feedback signal from the cable 150 in response to the signal. The hub 170 may transmit the recognition information to the image processing device 600. In addition, the hub 170 may transmit the recognition information to the scanner 100.

When the recognition information indicates that the first cable 150 is not genuine, the hub 170 may record log information about the first cable 150 in a memory. In addition, the hub 170 may turn off a power of the power supply device.

When the recognition information indicates that the first cable 150 is not genuine, the scanner 100 may record log information about the first cable 150 in the memory. In addition, the scanner 100 may control a preset function of the scanner 100 or may perform an operation of controlling a voltage booster circuit for increasing a voltage of the scanner 100.

In addition, at a third location 530, the hub 170 may perform an operation of identifying whether the second cable 190 is genuine. Specifically, the hub 170 may transmit a signal to the second cable 190. The hub 170 may obtain recognition information indicating whether the second cable 190 is genuine based on a feedback signal from the second cable 190 in response to the signal. The hub 170 may transmit the recognition information to the image processing device 600.

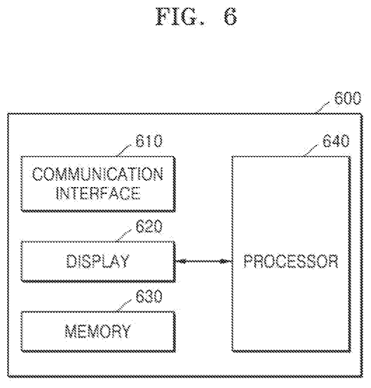

When the recognition information indicates that the first cable 150 or the second cable 190 is not genuine, the image processing device 600 may display notification information indicating the use of a non-genuine cable. A configuration and detailed operations of the image processing device 600 are described with reference to FIGS. 6 to 8C.

FIG. 6 is a block diagram illustrating a configuration of an image processing device according to an embodiment.

Referring to FIG. 6, an image processing device 600 may include a communication interface 610, a display 620, a memory 630, and a processor 640. However, not all of the shown elements are essential elements. The image processing device 600 may be implemented by more elements than shown, and the image processing device 600 may be implemented by less elements than shown. Hereinafter, the elements described above are discussed.

The communication interface 610 may perform communication with an external device. Specifically, the communication interface 610 may be connected to a network via a wire or wirelessly, and may perform communication with the external device. Here, the external device may be a server, a smartphone, a tablet, a PC, the scanner 100, or the like.

The communication interface 610 may include a communication module for supporting one of various wired and wireless communication schemes. For example, the communication module may be in the form of a chipset, or may be a sticker/barcode (e.g., a sticker including an NFC tag) including information necessary for communication. In addition, the communication module may be a short-distance communication module or a wired communication module.

For example, the communication interface 610 may support at least one of Wireless LAN, Wi-Fi, WFD, Bluetooth, BLE, Wired Lan, NFC, Zigbee, IrDA, 3G, 4G, and 5G.

The display 620 may display information, such as a result according to a control or processing of an operation of the image processing device 600. The display 620 may correspond to one of a liquid crystal display, a thin-film transistor-liquid crystal display, an organic light-emitting diode, a flexible display, a three-dimensional (3D) display, and an electrophoretic display. When the display 620 is configured in a touch screen, the display 620 may also be used as an input device to which information according to a user's touch may be input.

The memory 630 may store software or a program. For example, the memory 630 may store a program for generating, editing, and managing image data received from the scanner 100. In addition, the memory 630 may store at least one instruction for receiving, from the scanner 100, recognition information indicating whether the cable is genuine, and executing an operating method of the image processing device 600 that displays notification information of a non-genuine product, based on the recognition information

The processor 640 may control overlap operations of the image processing device 600 and may include at least one processor, such as a CPU and the like. The processor 640 may include at least one specialized processor corresponding to each function, or may be a processor in a single integrated form.

The processor 640 may execute a program stored in the memory 630, read data or a file stored in the memory 630, or store a new file in the memory 630. The processor 640 may execute the instructions stored in the memory 630.

The processor 640 may receive, from the scanner 100 via the communication interface 610, recognition information whether a cable connected to the scanner 100 is genuine. When the recognition information indicates that the cable is not genuine, the processor 640 may display, on the display 620, notification information indicating that the cable is not genuine. For example, the notification information may include at least one from among information indicating that the cable is non-genuine and information for requesting replacement of the cable with a genuine one.

In addition, the processor 640 may receive, via the communication interface 610, image data of an object obtained from the scanner 100. The processor 640 may display, on the display 620, an image corresponding to the image data of the object. The processor 640 may display the notification information on the image.

For example, the processor 640 may control the display 620 to perform at least one operation from among an operation of displaying the notification information in a pop-up window, an operation of displaying the notification information in an area other than an area of interest included in the image corresponding to the image data of the object, and an operation of displaying a watermark indicating the notification information.

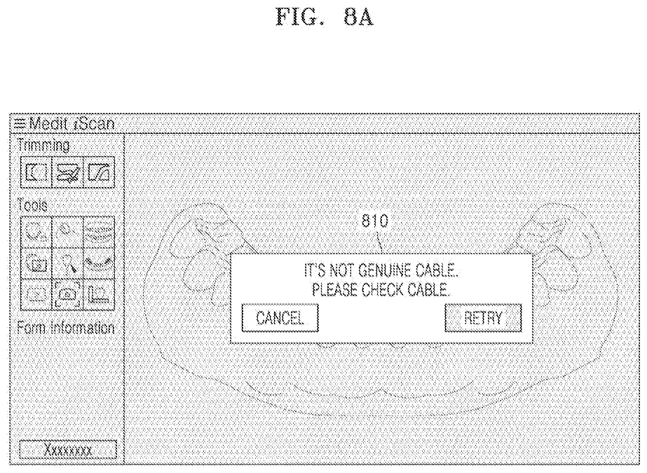

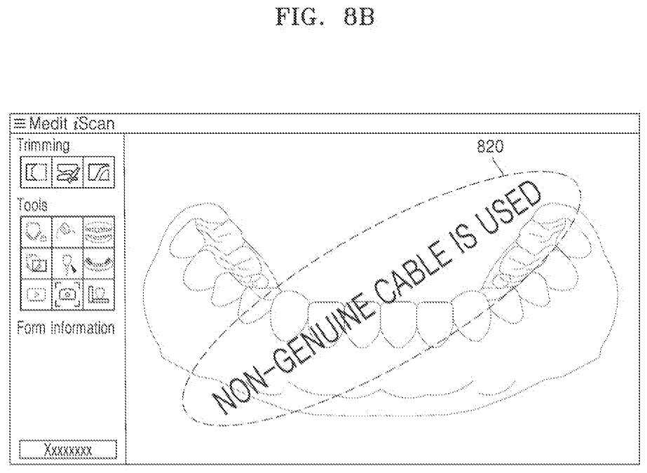

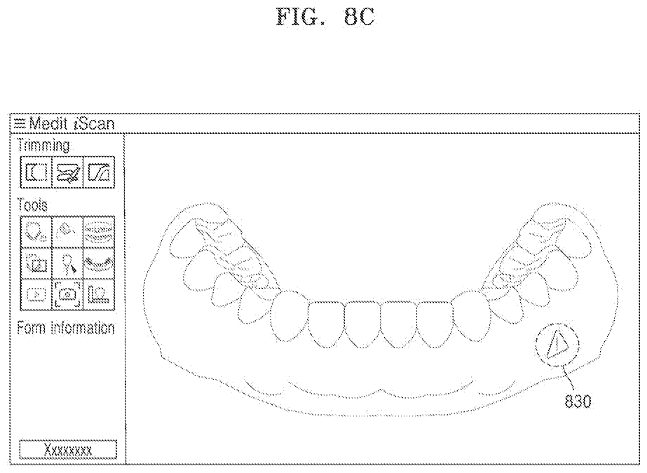

For example, the display 620 may display, in a pop-up window, notification information indicating that the cable is not genuine. For example, the pop-up window may indicate a window additionally displayed on an execution screen of a program of the scanner 100. An embodiment in which the notification information is displayed in a pop-up window is described with reference to FIG. 8A.

For example, the display 620 may display the notification information indicating that the cable is not genuine, as a watermark. An embodiment in which the notification information is displayed as a watermark is described with reference to FIG. 8B.

For example, the display 620 may display an image of an object. The display 620 may display notification information in an area other than an area of interest within the image. For example, the notification information may be displayed as a mark indicating that the cable is not genuine. For example, the mark may be expressed in a character or a figure. An embodiment in which the notification information is displayed in an area other than an area of interest is described with reference to FIG. 8C.

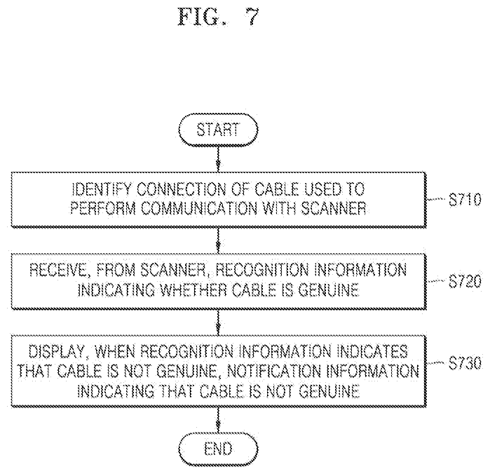

FIG. 7 is a flowchart of an operating method of an image processing device, according to an embodiment.

Referring to FIG. 7, in operation S710, the image processing device 600 may confirm connection of a cable used to perform communication with the scanner 100. For example, the cable may be directly connected to the scanner 100, and may be directly or indirectly connected to the image processing device 600.

In operation S720, the image processing device 600 may receive, from the scanner 100, recognition information indicating whether the cable is genuine.

In addition, when the genuineness of the cable is identified by the hub 170 described with reference to FIG. 5, the image processing device 600 may receive, from the hub 170, the recognition information indicating whether the cable is genuine. Here, the cable may be the first cable 150 connected to the scanner 100 and the hub 170, or the second cable 190 connected to the hub 170 and the image processing device 600.

In operation S730, when the recognition information indicates that the cable is not genuine, the image processing device 600 may display notification information indicating that the cable is not genuine.

For example, the notification information may include at least one from among information indicating that the cable is non-genuine and information for requesting replacement of the cable with a genuine one.

For example, the image processing device 600 may receive, from the scanner 100, image data of an object obtained from the scanner 100. The image processing device 600 may display the notification information on an image corresponding to the image data.

For example, the image processing device 600 may display the notification information in a pop-up window. In addition, the image processing device 600 may display the notification information in an area other than an area of interest included in the image corresponding to the image data of the object. In addition, the image processing device 600 may display a watermark indicating the notification information.

FIGS. 8A to 8C illustrate screens on which notification information indicating that the cable is not genuine is displayed when the cable is identified to be non-genuine, according to an embodiment.

Referring to FIG. 8A, when the recognition information received from the scanner 100 indicates that the cable is not genuine, the image processing device 600 may display notification information indicating that the cable is not genuine.

For example, the image processing device 600 may perform control such that an execution screen of a program for generating, editing, and managing the image corresponding to the image is deactivated, and display the notification in a pop-up window. For example, the deactivated execution screen of the program may be a screen in a state in which a user is not capable of controlling a function of the program. For example, the deactivated execution screen of the program may be a screen in a state of being shaded so that information in the screen may not be identified.

As shown in FIG. 8A, the image processing device 600 may display a pop-up window 810 indicating that the cable connected to the scanner 100 and the image processing device 600 is not a genuine cable. In addition, the image processing device 600 may display a pop-up window for requesting replacement of the cable. For example, until the cable is replaced with a genuine cable, the image processing device 600 may control an execution screen of the program to be deactivated or to be activated only for a preset time. In addition, the image processing device 600 may display phrases, such as “The cable currently connected to the scanner is not a genuine cable provided by Medit. When a cable other than a genuine cable is used, scanning is possible, but some functions may be limited. In case of problems during use, please use the gray cable included in the product package. The use of cables other than genuine cables is not guaranteed by Medit.”

Referring to FIG. 8B, the image processing device 600 may display an image corresponding to image data received from the scanner 100. To indicate that the cable is not a genuine cable, the image processing device 600 may display, on a screen on which the image is displayed, a watermark 820 indicating notification information.

For example, the watermark 820 may be expressed in one of a preset character and a figure. In addition, at least one piece of style information from among a location, size, and sharpness of the watermark 820 may be set in advance. In addition, the style information of the watermark 820 may be changed based on input information of a user.

For example, until before the cable is replaced with a genuine cable, the image processing device 600 may display the watermark 820 on the display. Even when an execution screen displayed on the display is changed, the watermark 820 may be displayed. For example, when the cable is replaced with a genuine cable, the image processing device 600 may remove the watermark 820 displayed on the screen.

Referring to FIG. 8C, the image processing device 600 may display an image corresponding to image data received from the scanner 100. For example, the image processing device 600 may display notification information in an area other than an area of interest included in the image. For example, the area of interest may be tooth, and the area other than the area of interest may be an area other than the tooth. For example, the image processing device 600 may display, in a gingiva area, a mark 830 indicating that the cable is not a genuine cable.

For example, until before the cable is replaced with a genuine cable, an image displayed on the image processing device may include the mark 830. In each image, the mark 830 may be displayed in an area other than the area of interest. For example, when the cable is replaced with a genuine cable, the image processing device 600 may generate and display an image from which the mark 830 is removed.

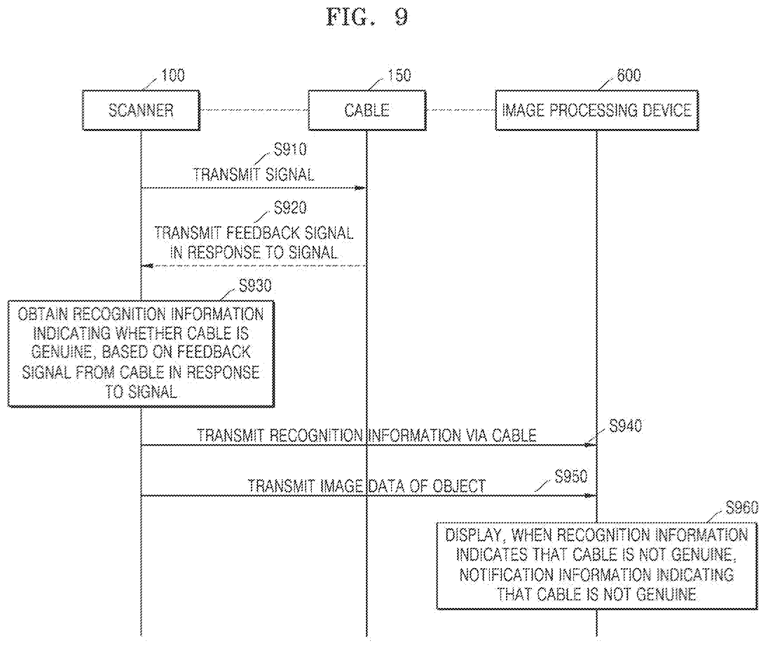

FIG. 9 is a flowchart of an operating method of an image processing system, according to an embodiment.

Referring to FIG. 9, in operation S910, the scanner 100 may transmit a signal to the cable 150 connected to the scanner 100 and the image processing device 600.

When the cable is genuine, in operation S920, the cable 150 may receive the signal from the scanner 100 and transmit a feedback signal in response to the signal. On the other hand, when the cable 150 is non-genuine, the cable 150 may not perform an operation according to operation S920.

In operation S930, the scanner 100 may obtain recognition information indicating whether the cable 150 is genuine, based on the feedback signal from the cable 150 in response to the signal.

For example, when the feedback signal is received from the cable 150, the scanner 100 may obtain verification information indicating that the cable 150 is identified to be genuine. On the other hand, when a feedback signal is not received from the cable 150, the scanner 100 may obtain error information indicating that the cable 150 is non-genuine.

For example, when the cable 150 is identified to be non-genuine, the scanner 100 may store log information about the cable 150. For example, the log information about the cable 150 may include at least one from among information on a time during which the cable 150 is connected and information on a time during which connection of the cable 150 is released.

For example, when the cable 150 is identified to be non-genuine, the scanner 100 may perform at least one operation from among an operation of controlling a preset function, an operation of controlling a voltage booster circuit for increasing a voltage of the scanner 100, and an operation of controlling at least one element of the scanner 100.

In operation S940, the scanner 100 may transmit the recognition information to the image processing device 600 via the cable 150. In operation S9509, the scanner 100 may transmit image data of an object to the image processing device 600.

In operation S960, when the recognition information indicates that the cable 150 is not genuine, the image processing device 600 may display notification information indicating that the cable 150 is not genuine.

For example, the image processing device 600 may display the notification information in a pop-up window. In addition, the image processing device 600 may display the notification information in an area other than an area of interest included in an image corresponding to the image data of the object. In addition, the image processing device 600 may display a watermark indicating the notification information.

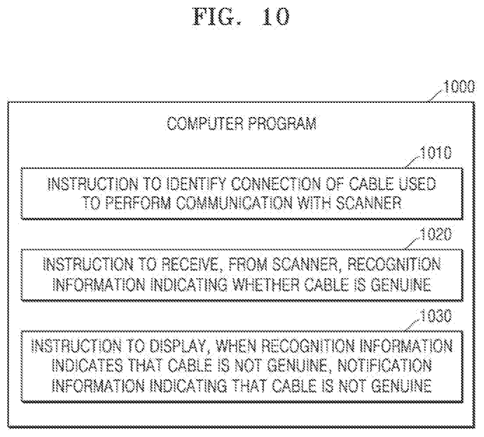

FIG. 10 is a diagram for describing instructions stored in a computer program stored in a computer-readable medium, according to an embodiment.

Referring to FIG. 10, a computer program 1000 may be stored in a computer-readable medium. The computer-readable medium may be combined with the image processing device 600, and the image processing device 600 may execute a computer program 1000.

For example, the computer program 1000 may store an instruction 1010 that confirms connection of a cable used to perform communication with a scanner.

For example, the computer program 1000 may store an instruction 1020 for receiving, from the scanner, recognition information indicating whether the cable is genuine.

For example, the computer program 1000 may, when recognition information indicates that the cable is not genuine, store an instruction 1030 for displaying notification information indicating that the cable is not genuine. For example, the computer program 1000 may store an instruction for receiving image data of an object obtained from the scanner 100, and an instruction for displaying notification information on an image corresponding to the image data.

An operating method of the scanner 100 or an operating method of the image processing device 600, according to an embodiment of the disclosure may be implemented in the form of program commands that may be performed through various computer means, and recorded in a computer-readable medium. In addition, an embodiment of the present disclosure may be a computer-readable recording medium on which one or more programs are recorded, the one or more programs including instructions for executing an operating method of a scanner or instructions for executing an operating method of an image processing device.

The computer-readable medium may include a program command, a data file, a data structure, etc. alone or in combination. The program command recorded on the medium may be those specially designed and configured for the present disclosure, or may be those well-known and available to a person having an ordinary skill in computer software. Examples of a computer-readable recording medium include magnetic media, such as hard disks, floppy disks and magnetic tapes, optical media, such as CD-ROMs and DVDs, magneto-optical media, such as floptical disks, and hardware devices specially configured to store and perform program commands, such as ROM, RAM, and flash memory. Examples of a program command include high-level language codes that may be executed by a computer by using an interpreter or the like, as well as machine language codes such as those produced by a compiler.

Here, a machine-readable storage medium may be provided in the form of a non-transitory storage medium. Here, the expression “non-transitory” only denotes that the storage medium is a tangible device and does not include a signal (e.g., an electromagnetic wave), and the term does not distinguish a case where data is semi-permanently stored in the storage medium from a case where data is temporarily stored. For example, the “non-transitory storage medium” may include a buffer in which data is temporarily stored.

According to an embodiment, a method according to various embodiments disclosed in the present document may be included and provided in a computer program product. The computer program product may be traded between sellers and buyers as commodities. The computer program product may be distributed in the form of a machine-readable storage medium (e.g., a compact disc read only memory (CD-ROM)), or may be directly distributed online (e.g., downloaded or uploaded) through an application store (e.g., Play Store™) or between two user devices (e.g., smartphones). In a case of online distribution, at least a portion of a computer program product (e.g., a downloadable app) may be temporarily stored in machine-readable storage medium, such as a server of a manufacturer, a server of an application store, or memory of a relay server, or may be temporarily created.

Specifically, according to disclosed embodiments, a program for performing operations of transmitting a signal to a cable connected to an image processing device that receives image data image data obtained from a scanner, obtaining recognition information indicating whether the cable is genuine, based on a feedback signal from the cable in response to the signal, and transmitting the recognition information to the image processing device via the cable, or a recording medium having the program recorded thereon may be implemented.

According to disclosed embodiments, a program for performing operations of receiving, from a scanner, recognition information indicating whether a cable connected to the scanner is genuine, and when the recognition information indicates that the cable is not genuine, displaying, on the display, notification information indicating that the cable is not genuine, or a recording medium having the program stored thereon may be implemented.

Although the embodiments are described above in detail, the scope of protection of the present disclosure is not limited thereto, and various modifications and improvement forms of a person having an ordinary skill in the technical field using the basic concept of the present disclosure defined by the following claims also belong to the protection scope of the present disclosure.

1. A scanner comprising:
a camera for obtaining image data by scanning an object;
a communication interface including a port to which a cable for performing communication with an external device is connected;
a processor; and a memory storing instructions executable by the processor,
wherein the processor is configured to, by executing the instructions:
transmit a signal to the cable connected to an image processing device which receives the image data; and
obtain recognition information indicating whether the cable is genuine, based on a feedback signal from the cable in response to the signal.

2. The scanner of claim 1, wherein the processor is further configured to, by executing the instructions:
based on the feedback signal being received from the cable, obtain verification information indicating that the cable is identified to be genuine; and
based on the feedback signal being not received from the cable, obtain error information indicating that the cable is identified to be non-genuine.

3. The scanner of claim 1, wherein the processor is further configured to, by executing the instructions, based on the cable being identified to be non-genuine, record log information about the cable in the memory.

4. The scanner of claim 3, wherein the log information includes history information about performing scanning using a non-genuine cable.

5. The scanner of claim 1, wherein the processor is further configured to, by executing the instructions, based on the cable being identified to be non-genuine, at least one operation from among an operation of controlling a preset function of the scanner, an operation of controlling a voltage booster circuit for boosting a voltage of the scanner, and an operation of controlling at least one element within the scanner.

6. The scanner of claim 1, further comprising a genuine cable connectable to the port,
wherein the genuine cable includes an identifier in response to a signal transmitted via the communication interface.

7. The scanner of claim 1, wherein the processor is further configured to, by executing the instructions:
obtain, from a power supply device for supplying power to the scanner, the recognition information indicating whether a first cable which connects the port to a first port of the power supply device, is genuine; and
transmit the recognition information to the image processing device via a second cable connecting a second port of the power supply device to the image processing device.

8. The scanner of claim 1, wherein the processor is further configured to, by executing the instructions, perform at least one operation from among an operation of transmitting the recognition information to the image processing device via the cable and an operation of outputting the recognition information.

9. An operating method of a scanner, the operating method comprising:
transmitting a signal to a cable connected to an image processing device which receives image data obtained from the scanner; and
obtaining recognition information indicating whether the cable is genuine, based on a feedback signal from the cable in response to the signal.

10. The operating method of claim 9, wherein the obtaining of the recognition information based on the feedback signal includes:
based on the feedback signal being received, obtaining verification information indicating that the cable is identified to be genuine; and
based on the feedback signal being not received, obtaining error information indicating the cable is identified to be non-genuine.

11. The operating method of claim 9, further comprising based on the cable being identified to be non-genuine, recording log information about the cable,
wherein the log information includes at least one from among information on a time during which the cable is connected and information on a time during which connection of the cable is released.

12. The operating method of claim 9, further comprising, based on the cable being identified to be non-genuine, performing at least one operation from among an operation of controlling a preset function of the scanner, an operation of controlling a voltage booster for boosting a voltage of the scanner, and an operation of controlling at least one element within the scanner.

13. A computer program stored in a computer-readable medium so as to be coupled to an image processing device and executed, the computer program comprising:
an instruction to identify connection of a cable used to perform communication with a scanner;
an instruction to receive, from the scanner, recognition information indicating whether the cable is genuine; and
an instruction to display, when the recognition information indicates that the cable is not genuine, notification information indicating that the cable is not genuine.

14. The computer program of claim 13, further comprising:
an instruction to receive image data of an object obtained from the scanner; and
an instruction to display the notification information on an image corresponding to the image data.

15. The computer program of claim 13, wherein the instruction to display the notification information includes:
at least one instruction from among an instruction to display the notification information in a pop-up window, an instruction to display the notification information in an area other than an area of interest included in an image corresponding to image data of an object, and at least one instruction from among instructions to display a watermark indicating the notification information.

16. The computer program of claim 13, wherein the notification information includes at least one from among information indicating that the cable is non-genuine and information indicating that replacement of the cable is requested with a genuine cable.
